(19) United States
(12) Patent Application Publication
(10) Pub. No.: US 2014/0358202 A1
(43) Pub. Date: Dec. 4, 2014

(54) Clogged Milk Duct Relief Device

(71) Applicant: Jaclyn L. Zanella, Lower Burrell, PA (US)

(72) Inventor: Jaclyn L. Zanella, Lower Burrell, PA (US)

(21) Appl. No.: 13/904,193

(22) Filed: May 29, 2013

(51) Int. Cl. A61F 7/08

(52) U.S. Cl. CPC A61F 7/08
USPC 607/108

(57) ABSTRACT

A clogged milk duct relief device provides warmth and vibration to the breast to relieve pain associated with a clogged milk duct. The device includes a pouch having an outer shell enveloping an interior space. A heat retention substance is positioned in the interior space of the pouch wherein the pouch is configured to deliver heat to a breast positioned against the pouch. A panel is coupled to the pouch proximate a perimeter edge of the panel such that the panel defines a pocket coupled to the pouch. A vibrator is selectively positionable in the pocket wherein the pouch is configured for delivering vibration to the breast.

### BACKGROUND OF THE DISCLOSURE

### Field of the Disclosure

The disclosure relates to lactation pain relief devices and more particularly pertains to a new lactation pain relief device for providing warmth and vibration to the breast to relieve pain associated with a clogged milk duct.

### SUMMARY OF THE DISCLOSURE

An embodiment of the disclosure meets the needs presented above by generally comprising a pouch having an outer shell enveloping an interior space. A heat retention substance is positioned in the interior space of the pouch wherein the pouch is configured to deliver heat to a breast positioned against the pouch. A panel is coupled to the pouch proximate a perimeter edge of the panel such that the panel defines a pocket coupled to the pouch. A vibrator is selectively positionable in the pocket wherein the pouch is configured for delivering vibration to the breast.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a partial cut-away top front side perspective view of a clogged milk duct relief device according to an embodiment of the disclosure.

FIG. 2 is a bottom view of an embodiment of the disclosure.

FIG. 3 is a side view of an embodiment of the disclosure.

FIG. 4 is a front view of an embodiment of the disclosure.

FIG. 5 is a bottom view of an embodiment of the disclosure.

### DESCRIPTION OF THE PREFERRED EMBODIMENT

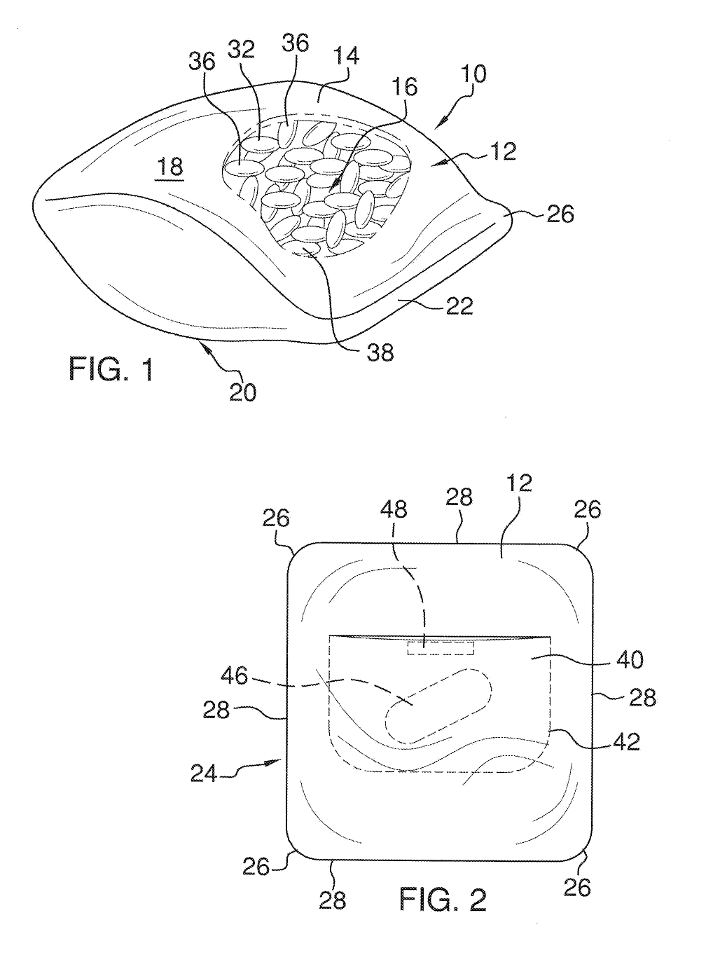

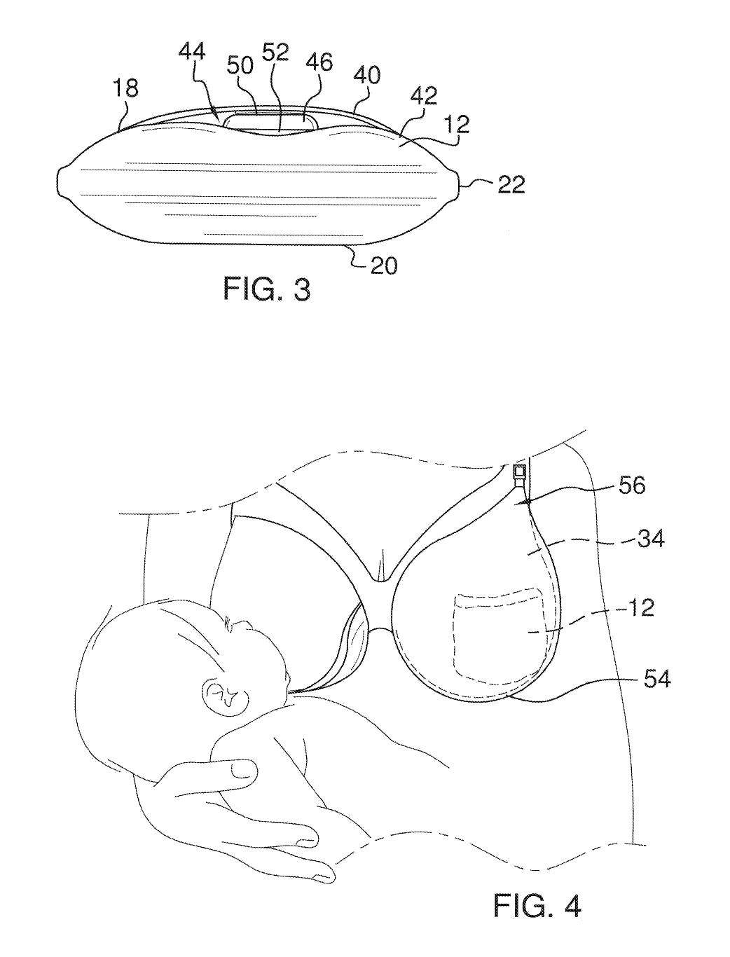

With reference now to the drawings, and in particular to FIGS. 1 through 5 thereof, a new lactation pain relief device embodying the principles and concepts of an embodiment of the disclosure and generally designated by the reference numeral 10 will be described.

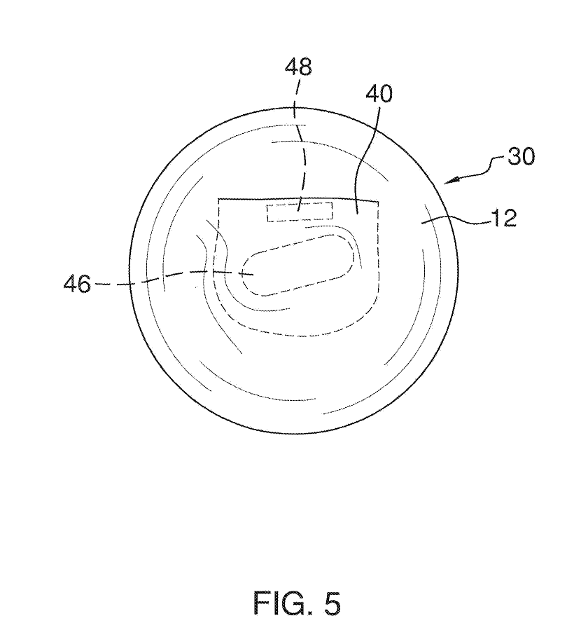

As best illustrated in FIGS. 1 through 5, the clogged milk duct relief device 10 generally comprises a pouch 12 having an outer shell 14 enveloping an interior space 16. The outer shell 14 has an upper surface 18, a lower surface 20, and a perimeter edge 22 extending between the upper surface 18 and the lower surface 20. The perimeter edge 22 may define a substantially rectangular shape 24 having a plurality of rounded corner sections 26 positioned between associated straight sections 28. Alternatively, the perimeter edge 22 may define a round shape 30 as shown in FIG. 5.

A heat retention substance 32 is positioned in the interior space 16 of the pouch 12 wherein the pouch 12 is configured to deliver heat to a breast 34 positioned against the pouch 12. The substance 32 may be a gel, or may be granular such as a plurality of grains 36 of rice 38, flax seed, or the like to conform to the shape of the breast 34 to transfer heat efficiently and comfortably to the breast 34. The substance 32 may be of the type conventionally heated in a microwave and retaining heat for an extended period of time thereafter.

A panel 40 is coupled to the pouch 12 proximate a perimeter edge 42 of the panel 40 by stitching or another conventional means such as adhesive or the like wherein the panel 40 defines a pocket 44 coupled to the pouch 12 on the outer shell 14. A vibrator 46 is selectively positionable in the pocket 44 wherein the pouch 12 is configured for delivering vibration to the breast 34. A closure 48 may he coupled to the panel 40. The closure 48 is selectively couplable to the outer shell 14 of the pouch 12 wherein the vibrator 46 is inhibited from inadvertent removal from the pocket 44. The closure 48 may be a portion of hook and loop fastener 50 and a complimentary second portion of hook and loop fastener 52 may be provided coupled to the outer shell 14 of the pouch 12 to secure the pocket 44 in a closed position.

In use, the pouch 12 is placed in a microwave to heat the substance 32. The vibrator 46 is activated and positioned in the pocket 44. The pouch 12 may then be positioned in a cup 54 of a bra 56 hold the pouch 12 against the breast 34, particularly in a position covering a nipple of the breast 34. The pouch 12 may be retained in the desired position by friction within the cup 54 of the bra 56. An additional retainer may also be employed to secure the pouch 12 relative to the cup 54 of the bra 56 to maintain the desired positioning.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements,

I claim:

1. A clogged milk duct relief device comprising:
a pouch having an outer shell enveloping an interior space;
a heat retention substance positioned in said interior space of said pouch wherein said pouch is configured to deliver heat to a breast positioned against said pouch;
a panel coupled to said pouch proximate a perimeter edge of said panel wherein said panel defines a pocket coupled to said pouch; and
a vibrator selectively positionable in said pocket wherein said pouch is configured for delivering vibration to the breast.

2. The device of claim 1, further comprising said heat retention substance being granular.

3. The device of claim 2, further comprising said heat retention substance being a plurality of grains of rice.

4. The device of claim 1, further comprising a closure coupled to said panel, said closure being selectively couplable to said pouch wherein said vibrator is inhibited from inadvertent removal from said pocket.

5. The device of claim 4, further comprising said closure being a portion of hook and loop fastener.

6. The device of claim 1, further comprising said outer shell having an upper surface, a lower surface, and a perimeter edge extending between said upper surface and said lower surface.

7. The device of claim 6, further comprising said perimeter edge defining a substantially rectangular shape.

8. The device of claim 7, further comprising said perimeter edge having a plurality of rounded corner sections positioned between associated straight sections.

9. The device of claim 6, further comprising said perimeter edge defining a round shape.

10. A clogged milk duct relief device comprising:
a pouch having an outer shell enveloping an interior space, said outer shell having an upper surface, a lower surface, and a perimeter edge extending between said upper surface and said lower surface;
a heat retention substance positioned in said interior space of said pouch wherein said pouch is configured to deliver heat to a breast positioned against said pouch, said heat retention substance being granular, said heat retention substance being a plurality of grains of rice;
a panel coupled to said pouch proximate a perimeter edge of said panel wherein said panel defines a pocket coupled to said pouch;
a vibrator selectively positionable in said pocket wherein said pouch is configured for delivering vibration to the breast; and
a closure coupled to said panel, said closure being selectively couplable to said pouch wherein said vibrator is inhibited from inadvertent removal from said pocket, said closure being a portion of hook and loop fastener.

11. The device of claim 10, further comprising said perimeter edge defining a substantially rectangular shape, said perimeter edge having a plurality of rounded corner sections positioned between associated straight sections.

12. The device of claim 10, further comprising said perimeter edge defining a round shape.
